(19) United States
(12) Patent Application Publication
(10) Pub. No.: US 2022/0091959 A1
(43) Pub. Date: Mar. 24, 2022

(54) METHOD AND SYSTEM FOR MONITORING ANTI-MICROBIAL EFFECTIVENESS OF AN ELECTRONIC DEVICE SCREEN PROTECTOR EMBEDDED WITH ANTI-MICROBIAL ADDITIVES

(71) Applicant: DJP Holdings Inc., Markham (CA)

(72) Inventor: Mohit Kripalani, Markham (CA)

(21) Appl. No.: 17/027,488

(22) Filed: Sep. 21, 2020

(51) Int. Cl. G06F 11/34; A61P 31/04; G06F 1/16

(52) U.S. Cl. CPC G06F 11/3423; G06F 2200/1634; G06F 1/1626; A61P 31/04

(57) ABSTRACT

The invention generally relates to a method and system for monitoring anti-microbial effectiveness of an electronic device screen protector embedded with anti-microbial additives. The method and system monitors one or more parameters that the electronic device is subjected to in daily use using a plurality of sensors. Further, the method and system tracks an activity level of a user on the electronic device screen protector using a plurality of electronic enumerators and counters. The method and system then quantifies the activity level and the one or more parameters of the electronic device screen protector into a measure of efficacy of the embedded anti-microbial additives on the electronic device screen protector and determines if the measure of efficacy exceeds a predetermined threshold. Upon determining the measure of efficacy exceeds the predetermined threshold, the method and system notifies the user of the electronic device to purchase a new screen protector.

### FIELD OF THE INVENTION

The invention generally relates to electronic devices such as, but not limited to, mobile phones, and multimedia tablets with tempered glass screen protectors embedded with anti-microbial additives. Specifically, the invention relates to a method and system for measuring anti-microbial efficacy/effectiveness of electronic device screen protectors embedded with anti-microbial additives, using sensors and wireless functionality integrated with the electronic devices.

### BACKGROUND OF THE INVENTION

Presently, anti-microbial additives embedded with different parts of a multimedia device or a shared device used in normal daily life are exposed to various weather conditions such as, but not limited to, a lot of light, humidity, moisture, dirt, oil, paraben and different temperatures which affect the efficacy of embedded anti-microbial additives that protect the device during daily usage.

Hitherto known electronic multimedia devices have a higher likelihood of being tainted by contaminants such as, but not limited to, bacteria and microbes during its daily use by a user or environmental exposure, as they lack an effective mechanism for enumerating its loss of efficacy.

Furthermore, the current state of sharing devices and mobility of modern multimedia devices make them prone to being exposed to the vagaries of the use and natural elements that reduce the effectiveness of the protection.

Moreover, traditional smartphones, mobile phones and other multimedia devices are not embedded with anti-microbial additives and therefore require an external protection such as, but not limited to, screen protectors, external cases to the multimedia devices, and protective covers which are vacuum sealed, all embedded with anti-microbial additives.

Currently available devices and external add-ons also lack sophisticated enumerators and counters to capture the information upon touch for providing information regarding attributes such as, but not limited to, a level of efficacy of the anti-microbial additive in the device, and a quality of the designed protection.

Therefore, in light of the above, there is a need for a method and system for determining anti-microbial efficacy of an electronic device screen protector embedded with anti-microbial additives, using sensors and wireless functionality integrated with the electronic device.

### BRIEF DESCRIPTION OF THE FIGURES

The accompanying figures where like reference numerals refer to identical or functionally similar elements throughout the separate views and which together with the detailed description below are incorporated in and form part of the specification, serve to further illustrate various embodiments and to explain various principles and advantages all in accordance with the invention.

FIG. 1 illustrates internal components of an electronic device for monitoring anti-microbial effectiveness of an electronic device screen protector embedded with anti-microbial additives in accordance with an embodiment of the invention.

FIG. 2 illustrates process flow and messages displayed on a user interface of the electronic device in accordance with an embodiment of the invention.

FIG. 3 illustrates a flow chart of a method for monitoring anti-microbial effectiveness of an electronic device screen protector embedded with anti-microbial additives in accordance with an embodiment of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION OF THE INVENTION

Before describing in detail embodiments that are in accordance with the invention, it should be observed that the embodiments reside primarily in combinations of method steps and system components for measuring anti-microbial efficacy/effectiveness of electronic device screen protectors embedded with anti-microbial additives, using sensors and wireless functionality integrated with the electronic devices.

Accordingly, the system components and method steps have been represented where appropriate by conventional symbols in the drawings, showing only those specific details that are pertinent to understanding the embodiments of the invention so as not to obscure the disclosure with details that will be readily apparent to those of ordinary skill in the art having the benefit of the description herein.

The terms “a” or “an”, as used herein, are defined as one or more than one. The term plurality, as used herein, is defined as two or more than two. The term another, as used herein, is defined as at least a second or more. The terms including and/or having, as used herein, are defined as comprising (i.e., open language). The term coupled, as used herein, is defined as connected, although not necessarily directly, and not necessarily mechanically. The terms program, software application, and the like as used herein, are defined as a sequence of instructions designed for execution on a computer system. A program, computer program, or software application may include a subroutine, a function, a procedure, an object method, an object implementation, an executable application, an applet, a servlet, a source code, an object code, a shared library/dynamic load library and/or other sequence of instructions designed for execution on a computer system.

Various embodiments of the invention disclose a method and system for monitoring anti-microbial effectiveness of an electronic device screen protector embedded with anti-microbial additives. The electronic device can be, but need not be limited to, a mobile phone, a multimedia tablet, phablet, and a smart phone. The screen protector can be, but need not be limited to, a tempered or a toughened glass or a protective guard, case, or cover. The anti-microbial additives can include, but need not be limited to, silver ion and copper.

The method and system monitors one or more parameters that the electronic device is subjected to in daily use, using a plurality of sensors. The one or more parameters can be, but need not be limited to, light, humidity, moisture, dirt, oil, paraben, different temperatures, pressure from touch and sweat and cleaning agents rubbed upon cleaning the electronic device screen protector regularly. The method and system also tracks an activity level of a user on the electronic device screen protector using a plurality of electronic enumerators and counters. In an instance, the activity level is a number of screen touches/holds. The method and system then quantifies the activity level and the one or more parameters into a measure of efficacy of the embedded anti-microbial additives on the electronic device screen protector. The measure of efficacy indicates either a loss/degradation or quality of the embedded anti-microbial additives. The method and system further determines if the measure of efficacy exceeds a predetermined threshold. Upon determining the measure of efficacy exceeds the predetermined threshold, the method and system notifies a user of electronic device.

In an embodiment, the method and system provides the user information related to the efficacy of the embedded anti-microbial additives on the electronic device screen protector.

In another embodiment, the method and system provides the user a message to purchase a new screen protector. Upon the user confirming purchase of a new screen protector via prompts presented at the user interface of the electronic device, a carrier is triggered and the carrier arranges shipment of the new screen protector.

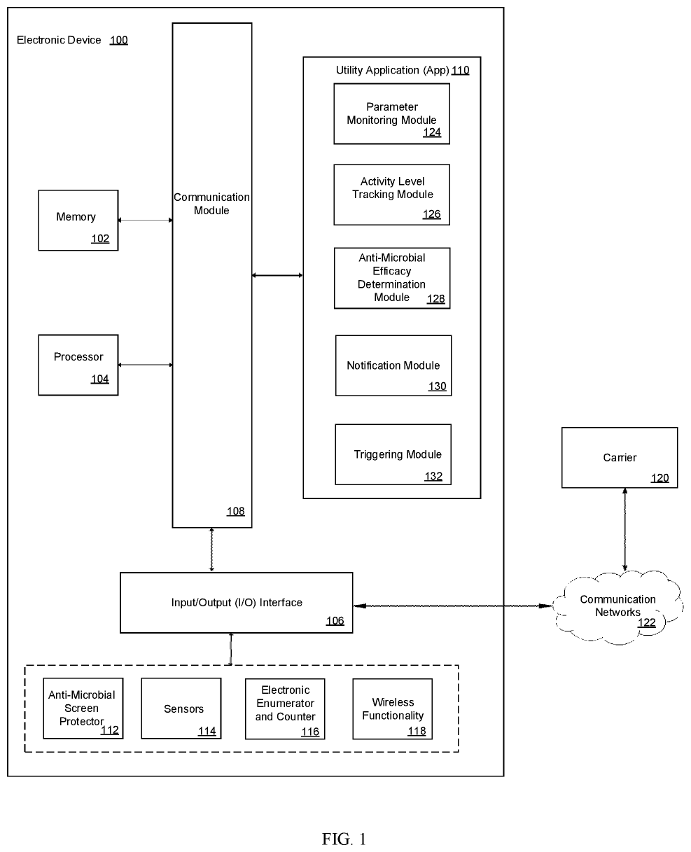

FIG. 1 illustrates internal components of an electronic device 100 for monitoring anti-microbial effectiveness of an electronic device screen protector embedded with anti-microbial additives in accordance with an embodiment of the invention.

Electronic device 100 can be, but need not be limited to, a mobile phone, a multimedia tablet, phablet, and a smart phone.

As illustrated in FIG. 1, electronic device 100, inter alia, comprises a memory 102 (such as, but not limited to, a non-transitory or a machine readable memory), a processor 104 (such as, but not limited to, a programmable electronic microprocessor, microcontroller, or similar device), and an input/output (I/O) interface 106.

I/O interface 106 may include a variety of software and hardware interfaces, for example, a web interface, a graphical user interface, and the like. I/O interface 106 may allow electronic device 100 interact with a user directly or through user devices. Further, I/O interface 106 may enable electronic device 100 to communicate with other user devices or computing devices, such as, but not limited to, web servers. I/O interface 106 can facilitate multiple communications within a wide variety of networks and protocol types, including wired networks, for example, local area network (LAN), cable, and the like, and wireless networks, such as wireless local area network (WLAN), cellular, or satellite. Further, I/O interface 106 may include one or more ports for connecting a number of devices to one another or to another server.

Processor 104 is communicatively coupled to memory 102 and I/O interface 106 via a communication module 108. Memory 102, processor 104, and I/O interface 106 further communicate with various components of electronic device 100 via communication module 108.

Communication module 108 may be configured to transmit data between modules, engines, databases, memories, and other components of electronic device 100 for use in performing the functions discussed herein. Communication module 108 may include one or more communication types and utilizes various communication methods for communication within electronic device 100.

Electronic device 100 further includes a utility application (app) 110 communicatively coupled to memory 102, processor 104, and I/O interface 106 via communication module 108. Utility app 110 is a software application executable by processor 104.

I/O interface 106 allows several components including, but not limited to, an anti-microbial screen protector 112, sensors 114, electronic enumerators and counters 116 and wireless functionality 118, to communicate with processor 104 and utility app 110.

In some instances, anti-microbial screen protector 112 is a protective case/guard embedded with anti-microbial additives such as, but not limited to, silver ion.

Sensors 114 include a plurality of sensors such as, but not limited to, tactile sensors (sensitive to touch, force, or pressure), contaminant sensors (for sensing presence of substances such as, but not limited to, contaminants, dirt, paraben, oil, cleaning agents, sweat), and environmental sensors (for sensing environmental conditions such as, but not limited to, weather, humidity, light, and temperature).

Electronic enumerators and counters 116 include algorithms for enumerating and keeping count of parameters such as, but not limited to, device usage, a number of touch interactions or touches/holds on anti-microbial screen protector 112. In accordance with an embodiment, electronic enumerators and counters 116 determine a weighted average of the number of touch interactions and abrasion on anti-microbial screen protector 112.

Wireless functionality 118 comprises wireless communication capabilities of electronic device 100 such as, but not limited to, Bluetooth, Wi-Fi, and Near-Field-Communication (NFC).

Electronic device 100 further communicates with a carrier 120 over one or more wired or wireless communication networks 122 via I/O interface 106. Portions of the communication networks 122 may be implemented using a wide area network, such as, but not limited to, the Internet, a local area network, a Wi-Fi or Bluetooth network, and combinations or derivatives thereof.

FIG. 1 further illustrates a schematic plan view showing the internal functional modules of utility app 110 that may be of various constructions and that which may use various communication types and protocols.

Utility app 110 includes a parameter monitoring module 124 which communicates and obtains information from plurality of sensors 114, for monitoring one or more parameters that electronic device 100 is subjected to in daily use. The one or more parameters can be, but need not be limited to, light, humidity, moisture, dirt, oil, paraben, different temperatures, pressure from touch and sweat and cleaning agents rubbed upon cleaning anti-microbial screen protector 112 regularly.

Utility app 110 further includes an activity level tracking module 126 which communicates with plurality of electronic enumerators and counters 116 for tracking an activity level on anti-microbial screen protector 112. In an instance, an activity level is a number of screen touches/holds.

Utility app 110 includes an anti-microbial efficacy determination module 128 for quantifying the activity level and the one or more parameters that were monitored into a measure of efficacy of the embedded anti-microbial additives on anti-microbial screen protector 112. The measure of efficacy indicates either a loss/degradation or quality of the embedded anti-microbial additives on anti-microbial screen protector 112. In an embodiment, anti-microbial efficacy determination module 128 determines efficacy of the embedded anti-microbial additives on anti-microbial screen protector 112 based on the weighted average of the number of touch interactions and abrasion on anti-microbial screen protector 112, obtained from electronic enumerators and counters 116.

Anti-microbial efficacy determination module 128 further determines if the measure of efficacy exceeds a predetermined threshold. Upon determining the measure of efficacy exceeds the predetermined threshold, a user of electronic device 100 is notified via a notification module 130.

In an instance, notification module 130 provides the user information related to the efficacy of the embedded anti-microbial additives on anti-microbial screen protector 112.

In another instance, notification module 130 provides the user a message to purchase a new screen protector. In this case, utility app 110 includes a triggering module 132 which triggers carrier 120 upon receiving a confirmation of purchase from the user. Carrier 120 then arranges shipment of the new screen protector.

In accordance with an exemplary implementation, the screen of electronic device 100 is divided into a plurality of grids, each grid having the area of a user's finger size for instance.

Electronic enumerators and counters 116 monitor a number of touch interactions of the user on each of the grid areas. In response to determining by anti-microbial efficacy determination module 128 that a number of touch interactions on a particular grid exceed a predetermined threshold, notification module 130 triggers a message to the user. For instance, anti-microbial efficacy determination module 128 determines that there is a deterioration in anti-microbial efficacy in a particular portion of anti-microbial screen protector 112, when a number of touch interactions on a corresponding grid area on the screen exceed a predetermined numerical value. Notification module 130 then notifies the user of the deterioration in efficacy and prompts the user with a message to purchase a new screen protector.

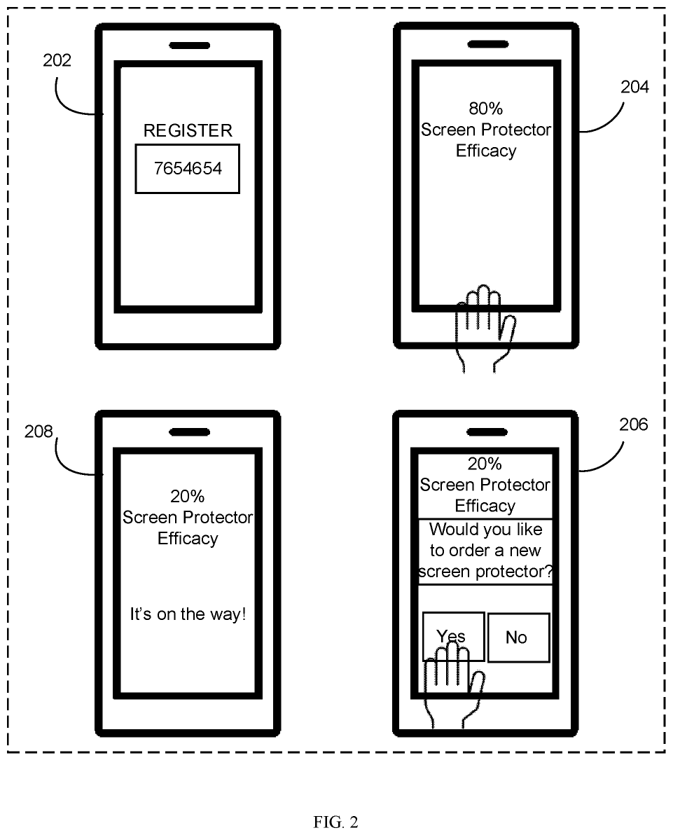

FIG. 2 illustrates process flow and messages displayed on a user interface of electronic device 100 in accordance with an embodiment of the invention.

Referring to FIG. 2, as depicted in 202, a user registers anti-microbial screen protector 102 in utility app 110 by entering a unique identifier or a serial number.

Utility app 110 then tracks antimicrobial abrasion in the background every time the user touches the screen, as depicted in 204.

Once anti-microbial screen protector 112 loses its efficacy (say the efficacy drops to 20%), utility app 110 prompts or notifies the user to purchase a new screen protector and receives either a confirmation of purchase or decline from the user, as depicted in 206.

If the user has confirmed purchase via the user interface of electronic device 100, carrier 120 arranges shipment and notifies the user, as depicted in 208.

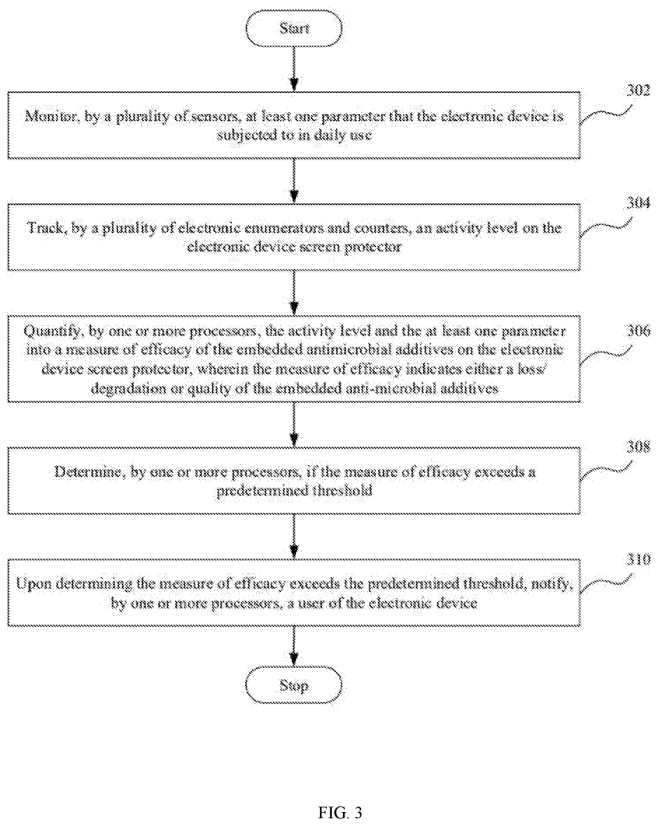

FIG. 3 illustrates a flow chart of a method for monitoring anti-microbial effectiveness of anti-microbial screen protector 112 of electronic device 100 embedded with anti-microbial additives in accordance with an embodiment of the invention.

As illustrated in FIG. 3, at step 302, parameter monitoring module 124 of utility app 110 communicates and obtains information from plurality of sensors 114, for monitoring one or more parameters that electronic device 100 is subjected to in daily use. The one or more parameters can be, but need not be limited to, light, humidity, moisture, dirt, oil, paraben, different temperatures, pressure from touch and sweat and cleaning agents rubbed upon cleaning anti-microbial screen protector 112 regularly.

At step 304, activity level tracking module 126 of utility app 110 communicates with plurality of electronic enumerators and counters 116 for tracking an activity level on anti-microbial screen protector 112. In an instance, an activity level is a number of screen touches/holds.

At step 306, anti-microbial efficacy determination module 128 of utility app 110 quantifies the activity level and the one or more parameters that were monitored into a measure of efficacy of the embedded anti-microbial additives on anti-microbial screen protector 112. The measure of efficacy indicates either a loss/degradation or quality of the embedded anti-microbial additives on anti-microbial screen protector 112.

At step 308, anti-microbial efficacy determination module 128 further determines if the measure of efficacy exceeds a predetermined threshold.

Upon determining the measure of efficacy exceeds the predetermined threshold, at step 310, a user of electronic device 100 is notified via notification module 130 of utility app 110.

In an instance, notification module 130 provides the user information related to the efficacy of the embedded anti-microbial additives on anti-microbial screen protector 112.

In another instance, notification module 130 provides the user a message to purchase a new screen protector. In this case, triggering module 132 of utility app 110 triggers carrier 120 upon receiving a confirmation of purchase. Carrier 120 then arranges shipment of the new screen protector.

The present invention is advantageous in that it provides a utility app which captures the efficacy of the anti-microbial properties. The app enumerates the number of touches and holds and uses an algorithm to encapsulate the different parameters that the electronic device is subjected to in daily use.

Further, the utility app within the electronic device communicates with a plurality of sensors to identify the efficacy or the loss of anti-microbial effectiveness in protection, and a level of the anti-microbial effectiveness, and further detects the quality of the embedded anti-microbial additives using the sensors. This information is then communicated to the user via the utility application.

Those skilled in the art will realize that the above recognized advantages and other advantages described herein are merely exemplary and are not meant to be a complete rendering of all of the advantages of the various embodiments of the present invention.

The system, as described in the invention or any of its components may be embodied in the form of a computing device. The computing device can be, for example, but not limited to, a general-purpose computer, a programmed microprocessor, a micro-controller, a peripheral integrated circuit element, and other devices or arrangements of devices, which are capable of implementing the steps that constitute the method of the invention. The computing device includes a processor, a memory, a nonvolatile data storage, a display, and a user interface.

In the foregoing specification, specific embodiments of the present invention have been described. However, one of ordinary skill in the art appreciates that various modifications and changes can be made without departing from the scope of the present invention. Accordingly, the specification and figures are to be regarded in an illustrative rather than a restrictive sense, and all such modifications are intended to be included within the scope of the present invention.

What is claimed is:

1. A method for monitoring anti-microbial effectiveness of an electronic device screen protector embedded with anti-microbial additives, the method comprising:
monitoring, by a plurality of sensors, at least one parameter that the electronic device is subjected to in daily use;
tracking, by a plurality of electronic enumerators and counters, an activity level on the electronic device screen protector;
quantifying, by one or more processors, the activity level and the at least one parameter into a measure of efficacy of the embedded anti-microbial additives on the electronic device screen protector, wherein the measure of efficacy indicates either a loss/degradation or quality of the embedded anti-microbial additives;
determining, by one or more processors, if the measure of efficacy exceeds a predetermined threshold; and
upon determining the measure of efficacy exceeds the predetermined threshold, notifying, by one or more processors, a user of the electronic device.

2. The method of claim 1, wherein a parameter is at least one of light, humidity, moisture, dirt, oil, paraben, different temperatures, pressure from touch and sweat and cleaning agents rubbed upon cleaning the screen protector regularly.

3. The method of claim 1, wherein an activity level is a number of screen touches/holds.

4. The method of claim 1, wherein the electronic device screen is divided into a plurality of grids.

5. The method of claim 4 comprises monitoring, by an electronic counter, a number of touch interactions on each grid of the plurality of grids and in response to the number of touch interactions on any grid exceeding a predetermined threshold, notifying a user of the electronic device.

6. The method of claim 1, wherein the notifying comprises providing to the user information related to the efficacy of the embedded anti-microbial additives on the electronic device screen protector.

7. The method of claim 1, wherein the notifying comprises providing to the user a message to purchase a new screen protector.

8. The method of claim 7 further comprises triggering a carrier upon receiving a confirmation of purchase, wherein the carrier arranges shipment of the new screen protector.

9. A system for monitoring anti-microbial effectiveness of an electronic device screen protector embedded with anti-microbial additives, the system comprising:
a memory;
a processor communicatively coupled to the memory, the processor configured to:
monitor, using a plurality of sensors, at least one parameter that the electronic device is subjected to in daily use;
track, using a plurality of electronic enumerators and counters, an activity level on the electronic device screen protector;
quantify the activity level and the at least one parameter into a measure of efficacy of the embedded anti-microbial additives on the electronic device screen protector, wherein the measure of efficacy indicates either a loss/degradation or quality of the embedded anti-microbial additives;
determine if the measure of efficacy exceeds a predetermined threshold; and
upon determining the measure of efficacy exceeds the predetermined threshold, notify a user of the electronic device.

10. The system of claim 9, wherein a parameter is at least one of light, humidity, moisture, dirt, oil, paraben, different temperatures, pressure from touch and sweat and cleaning agents rubbed upon cleaning the screen protector regularly.

11. The system of claim 9, wherein an activity level is a number of screen touches/holds.

12. The system of claim 9, wherein the electronic device screen is divided into a plurality of grids.

13. The system of claim 12, wherein the processor is configured to monitor, by an electronic counter, a number of touch interactions on each grid of the plurality of grids and in response to the number of touch interactions on any grid exceeding a predetermined threshold, notifying a user of the electronic device.

14. The system of claim 9, wherein the processor is configured to provide to the user information related to the efficacy of the embedded anti-microbial additives on the electronic device screen protector.

15. The system of claim 9, wherein the processor is configured to provide to the user a message to purchase a new screen protector.

16. The system of claim 15, wherein the processor is further configured to trigger a carrier upon receiving a confirmation of purchase, wherein the carrier arranges shipment of the new screen protector.
